(19) United States
(12) Patent Application Publication
(10) Pub. No.: US 2026/0198830 A1
(43) Pub. Date: Jul. 16, 2026

(54) DUAL-CONDUCTIVE FIBER NETWORK STRUCTURE MATERIAL AND PREPARATION METHOD AND APPLICATION THEREOF

(71) Applicants: Shenzhen Second People's Hospital (Shenzhen Institute of Translational Medicine), Shenzhen (CN); Tsinghua Shenzhen International Graduate School, Shenzhen (CN)

(72) Inventors: Lei Yang, Shenzhen (CN); Ruomei Shao, Shenzhen (CN); Wenyu Zhou, Shenzhen (CN); Shuqing Sun, Shenzhen (CN)

(21) Appl. No.: 19/439,545

(22) Filed: Jan. 5, 2026

(30) Foreign Application Priority Data
Jan. 10, 2025 (CN) 202510042611.0

(51) Int. Cl. A61B 5/263; D01F 1/09; D01F 6/14; D01F 6/18; D06M 11/55; D06M 101/30

(52) U.S. Cl. CPC A61B 5/263; D01F 1/09; D01F 6/14; D01F 6/18; D06M 11/55; A61B 2562/125; D06M 2101/30; D10B 2321/06; D10B 2321/10; D10B 2401/16

(57) ABSTRACT

Provided are a dual-conductive fiber network structure material and a preparation method and application thereof. The dual-conductive fiber network structure material, with a core of dual-phase spun fibers obtained from polyacrylonitrile fibers comprised of polyacrylonitrile and deprotonated polyaniline and chitosan gel fibers comprised of chitosan and polyvinyl alcohol (PVA), is cladded with a long-chain polyaniline shell, and can be stored for a long time. The dual-conductive fiber network structure material solves the problems of large signal-to-noise ratio and poor compliance and stability of electrodes of skin-contact devices in the prior art, and the preparation method solves the problem of difficulty in spinning the chitosan solution and the polyacrylonitrile-polyaniline solution in the prior art and reconciles the problems of stability of chitosan gel fibers and conductivity requirements of protonic acid-doped polyaniline.

### CROSS-REFERENCE TO RELATED APPLICATION

The present application claims priority from Chinese Patent Application No. 202510042611.0 filed on January 10, 2025, the contents of which are incorporated herein by reference in their entirety.

### FIELD OF INVENTION

The present invention relates to the field of flexible electronic materials, and in particular to a dual-conductive fiber network structure material and a preparation method and application thereof.

### BACKGROUND ART OF THE INVENTION

Electrophysiological signal acquisition devices with excellent skin contact conductance and adaptation to flexibility, air permeability and perspiration of skin have significant advantages in healthcare applications, and become the bottleneck problem for long-term continuous monitoring of the existing electromyographic sensing technology due to the fact that electromyographic signals are weak time-varying non-stationary signals, coupled with complex interface interference such as motion artifacts superimposed on the sensor skin interface by the dynamic process in the real world. Especially in the field of exercise rehabilitation and neural prosthetics: due to tissue fibrosis at amputation sites, muscle nerve atrophy poses a serious obstacle to the extraction of muscle nerve electrical signals; and the hyperplasia of the stratum corneum caused by other diseases, the development of sweat glands or the state of high-intensity exercise all pose new requirements and challenges for the detection of electrophysiological signals spanning the electrical interface of the skin.

In the prior art, the traditional electrode schemes mainly include wearable Ag/AgCl gel electrodes and metal stickers. Due to problems such as signal decay caused by solvent evaporation and electrolyte irritation to the skin, such gel electrodes are not suitable for long-term stable detection of bioelectrical signals, and in the measurement of muscle activation with a small amplitude below 10% MVC, the signals of such electrodes are unstable and have large noise; and the metal stickers are widely used in new consumer electronic devices such as commercial multimodal watches and bracelets. The metal stickers have poor compliance, and are greatly different from human skin in elastic modulus due to the fact that the elastic modulus of metals is usually close to 102 GPa. This problem of physical property mismatch causes severe damage to the stability of the skin electrode interface and is unable to resist the noise impact brought by body movement. In addition, even if the metal stickers are made into thin sheets, external pressure is also required to collect signals, and the metal stickers can be easily peeled off and cannot be stably attached.

Further enhancing the reliability and compliance of electrophysiological signal acquisition has become a concentrated research issue in recent years. Firstly, in terms of dry electrodes, for example, in well-known journals (RSC Adv., 2015, 5, 11627) (Nano Lett. 2019, 19,6853−6861), silver nanowire electrodes are published, the compliance is improved, but the thickness is relatively large (in millimeters or even centimeters) due to the limitations of the preparation method, and the skin electrode contact impedance is lower than that of the Ag/AgCl gel electrodes (the working impedance in the human electrophysiological frequency region is at an MΩ level). In terms of wet electrodes, for example, in top international journals (Nat. Commun., 2023, 14:6494 1) (Adv. Mater. 2020, 32, 2003723), the ionized gel electrodes enhance the signal-to-noise ratio and compliance of signal acquisition. However, gel-type array devices are usually prepared by a template method, with thicknesses ranging from hundreds of micrometers to even millimeters. When attached to the body surface for detection, the gel-type array devices have poor breathability and sweat permeability, and clog pores for a long time, which easily causes skin inflammatory response. Moreover, considerable challenges exist in the long-term stability and reliability of such electrode devices.

### DISCLOSURE OF THE INVENTION

In view of this, a technical problem to be solved by the present invention is to provide a dual-conductive fiber network structure material and a preparation method and application thereof. The dual-conductive fiber network structure material provided by the present invention has ultra-low skin interface impedance and good stability, and as an electrode of a skin contact device, has good adhesion and compliance to fine lines of human skin.

The present invention provides a dual-conductive fiber network structure material, comprising:

A first conductive fiber network, comprised of polyacrylonitrile, deprotonated polyaniline, chitosan and polyvinyl alcohol (PVA);

Sulfuric acid-doped polyaniline cladded on the first conductive fiber network.

The dual-conductive fiber network structure material provided by the present invention is a dual network core-shell structure fiber material. The first conductive fiber network as a core is a spinning fiber network where chitosan gel fibers and polyacrylonitrile fibers (polyacrylonitrile-chitosan dual-phase spun fibers for short) are uniformly distributed, wherein the chitosan gel fibers are comprised of chitosan and PVA, and the diameter of the chitosan gel fibers is about 10-50 nm; and the polyacrylonitrile fibers are comprised of polyacrylonitrile and deprotonated polyaniline, and the diameter of the polyacrylonitrile fibers is about 90-200 nm. The sulfuric acid-doped polyaniline as a shell is a long-chain polyaniline shell, and is cladded on the fibers that constitute the first conductive fiber network, thereby forming a second conductive layer network.

The dual-conductive fiber network structure material provided by the present invention is a flexible functional material, an electrode material and a fibrous porous material, with the thickness controlled to 1-50 μm, preferably 5-30 μm, and has air permeability, perspiration and good adhesion and compliance to fine lines of human skin. Based on the dual-conductive fiber network structure, the dual-conductive fiber network structure material can maintain high skin electrode conductance on the wet and dry surfaces of the skin, enhancing the signal-to-noise ratio of electrophysiological signal acquisition. Moreover, since the chitosan gel fibers and the polyacrylonitrile fibers containing polyaniline maintain a stable form for a long time, the dual-conductive fiber network structure material will not denature due to moisture or dryness and is suitable for long-term storage.

The present invention also provides a preparation method for the dual-conductive fiber network structure material, comprising the following steps:

S1) Electrospinning a first spinning solution and a second spinning solution at an exit spacing of 0.9-3.0 cm;

The first spinning solution comprises polyacrylonitrile and deprotonated polyaniline;

The second spinning solution comprises chitosan and PVA;

S2) After curing a material obtained in step S1) in an alkaline solution, cladding sulfuric acid-doped polyaniline on a surface of the material to obtain a dual-conductive fiber network structure material.

In the present invention, firstly, the first spinning solution and the second spinning solution are electrospun at an exit spacing of 0.9-3.0 cm, preferably 0.9-1.5 cm, and more preferably 0.9-1.1 cm. The present invention adopts a bicomponent conjugate spinneret design to electrospin the first spinning solution and the second spinning solution, that is, two spinning needles are placed side by side for electrospinning, and the spacing between spinneret nozzles of the two spinning needles is 0.9-3.0 cm, which can prevent the two spinning solutions from prematurely condensing due to mutual effect at the exit.

In the present invention, when the electrospinning is carried out, a spinning solution jet is dried with hot air at an angle of 20-50°. Specifically, when the electrospinning is carried out, the spinning solution jet sprayed from the spinneret nozzles of the spinning needles is dehumidified and dried with hot air at an angle of 20-50°, preferably 30-45°. In the present invention, when the electrospinning is carried out, the spinning solution jet is dried with gentle breeze so that the two solvents are timely and fully volatilized during the bicomponent conjugate spinneret spinning jet process.

The first spinning solution of the present invention comprises polyacrylonitrile and deprotonated polyaniline. During the electrospinning process in the present invention, deprotonated polyaniline particles are uniformly dispersed in the polyacrylonitrile spinning solution as seeds, which can subsequently guide the crystal growth of the long-chain polyaniline shell and enhance the binding degree with the fibers of the core (the first conductive fiber network). Specifically, the first spinning solution is obtained from a polyacrylonitrile solution with a polyacrylonitrile mass fraction of 5 wt%-20 wt% and a deprotonated polyaniline dispersion liquid with a deprotonated polyaniline mass fraction of 8 wt%-15 wt%, preferably a polyacrylonitrile solution with a polyacrylonitrile mass fraction of 8 wt%-12 wt% and a deprotonated polyaniline dispersion liquid with a deprotonated polyaniline mass fraction of 8 wt%-12 wt%, and more preferably a polyacrylonitrile solution with a polyacrylonitrile mass fraction of 10 wt% and a deprotonated polyaniline dispersion liquid with a deprotonated polyaniline mass fraction of 10 wt%; and a weight ratio of the deprotonated polyaniline dispersion liquid in the first spinning solution is 10%-50%, preferably 20%-30%. In some embodiments of the present invention, the first spinning solution is obtained by mixing a dimethyl formamide (DMF) solution of polyacrylonitrile and an N-methyl-2-pyrrolidone (NMP) of a deprotonated polyaniline dispersion liquid. A molecular weight Mv of the polyacrylonitrile of the present invention is 100000-500000, preferably 100000-200000, and more preferably 150000.

The second spinning solution of the present invention comprises chitosan and PVA. Specifically, the second spinning solution is obtained from a PVA solution and a chitosan solution with a chitosan mass fraction of 3 wt%-4 wt%, and preferably a PVA solution and a chitosan solution with a chitosan mass fraction of 3 wt%; and a weight ratio of the PVA solution to the chitosan solution is 0.5:(8-15), preferably 0.5:12.5. In some embodiments of the present invention, the PVA solution is an aqueous solution of the PVA; and the chitosan solution is an acetic acid solution of chitosan. The chitosan of the present invention is low-polymerization degree chitosan with a deacetylation degree greater than 99%. The PVA-1-1 of the present invention is PVA with a viscosity coefficient of 54-66 mPa.s. The present invention increases the proportion of chitosan, making the formed hydrogel-phase spinning resistant to strong alkali and sulfuric acid, and avoiding the possibility of hydrophilic network hydrolysis.

In the present invention, a first spinning solution and a second spinning solution are electrospun preferably at a volume ratio of 1:1. A spinning speed of the electrospinning of the present invention is 7-30 µL/min, and preferably 7-10 µL/min. A spinning voltage of the electrospinning of the present invention is 19-30 kV, and preferably 19-28 kV. The present invention can precisely control the thickness of the finally obtained dual-conductive fiber network structure material through the spinning time and the advancement speed of the spinning solutions, and can further adjust the pore size between the fibers of the finally obtained dual-conductive fiber network structure material through the positive high voltage of the electrospinning and the stretching speed of the receiving end roller.

The deprotonated polyaniline of the present invention is dedoped polyaniline, and is prepared by the following method: obtaining doped polyaniline from ammonium persulfate, hydrochloric acid and aniline, and deprotonating the doped polyaniline in aqueous ammonia to obtain the deprotonated polyaniline. Specifically, the deprotonated polyaniline is prepared by the following method: at room temperature, mixing a hydrochloric acid solution of aniline and a hydrochloric acid solution of ammonium persulfate, standing for 2-5 h, and carrying out filtering and drying to obtain doped polyaniline; and letting the doped polyaniline stand in saturated aqueous ammonia for 24-48 h, and carrying out filtering and drying to obtain the deprotonated polyaniline. In some embodiments of the present invention, the hydrochloric acid solution of aniline is obtained by dissolving 3.5-4.5 g of aniline in 40-60 mL of 0.8-1.2 M hydrochloric acid solution; the hydrochloric acid solution of ammonium persulfate is obtained by dissolving 12-13 g of ammonium persulfate in 40-60 mL of 0.8-1.2 M hydrochloric acid solution; and the aqueous ammonia is saturated aqueous ammonia.

In the present invention, after the first spinning solution and the second spinning solution are electrospun, the material obtained by electrospinning is cured in an alkaline solution, and sulfuric acid-doped polyaniline is cladded on a surface of the material to obtain a dual-conductive fiber network structure material. In the present invention, to reconcile the stability of chitosan gel fibers and conductivity requirements of protonic acid-doped polyaniline, the material obtained by electrospinning, i.e., polyacrylonitrile-chitosan dual-phase spun fibers, is cross-linked and reinforced with chitosan fibers in an alkaline solution, dilute sulfuric acid is selected as doped acid (chitosan is stable in dilute sulfuric acid), and a long-chain polyaniline shell is further cladded on the dual-phase spun fibers.

Specifically, the material obtained by electrospinning is cured in a sodium hydroxide solution, the cured material is stood in a mixed solution of ammonium persulfate, sulfuric acid and aniline at 0-2℃ to obtain a dual-conductive fiber network structure material. In some embodiments of the present invention, the material obtained by electrospinning is cured in a 1.8-2.2 M sodium hydroxide solution for 2-3 min; and the cured material is stood in a mixed solution of ammonium persulfate, sulfuric acid and aniline at 0-2℃ for 22-26 h. In this process, a long-chain sulfuric acid-doped polyaniline shell is formed on the fibers of the first conductive fiber network, and the long-chain sulfuric acid-doped polyaniline shell is the second conductive layer network, thus obtaining the dual-conductive fiber network structure material.

In some embodiments of the present invention, the mixed solution of ammonium persulfate, sulfuric acid and aniline is obtained by mixing 80-120 mL of 3 wt%-3.5 wt% ammonium persulfate solution, 80-120 mL of 10 wt%-12 wt% sulfuric acid solution and 40-60 mL of 1.5 wt%-2.5 wt% aniline solution at 0-2℃, and the solvents of the solutions are all 40%-60% ethanol aqueous solutions.

The preparation method for a dual-conductive fiber network structure material provided by the present invention realizes one-time forming through bicomponent conjugate spinneret electrospinning, with adjustable fineness and pore size, and can provide a more precise solution for physiological detection of wearable bioelectronics and deep electromyographic exercise. By strictly controlling the mutual permeability and smoothness of double networks, the possibility of affecting condensing by the two solutions in advance is avoided, and the difficulty that the first spinning solution and the second spinning solution are mutually insoluble, influence each other and are difficult to spin in the present invention is overcome; and on this basis, a long-chain polyaniline shell is cladded by in-situ polymerization with low-temperature oxygen reduction, and a long-chain polyacrylonitrile@polyaniline-chitosan@polyaniline dual network core-shell structure fiber material is finally prepared.

The present invention also provides application of the dual-conductive fiber network structure material or the dual-conductive fiber network structure material obtained by the preparation method in preparation of electrodes. The dual-conductive fiber network structure material provided by the present invention is used as an electrode, especially for skin-contact devices. Due to the electropositivity of chitosan and the conductive property of hydrogel, the adhesion between a sensor and the negative potential surface of the skin and the interfacial conductance are significantly improved. Meanwhile, the existence of long-chain polyaniline networks significantly increases the skin interfacial conductance of the sensor in the dry electrode state, and the overall skin electrode contact impedance is two orders of magnitude lower than that of commercial Ag/AgCl gel electrodes (in an electrophysiological signal response region). The dual-conductive fiber network structure material has good compliance and can significantly improve the signal-to-noise ratio of electromyographic signals with MVC below 10%.

The present invention provides a dual-conductive fiber network structure material and a preparation method and application thereof. The dual-conductive fiber network structure material provided by the present invention, with a core of dual-phase spun fibers obtained from polyacrylonitrile fibers comprised of polyacrylonitrile and deprotonated polyaniline and chitosan gel fibers comprised of chitosan and polyvinyl alcohol (PVA), is cladded with a long-chain polyaniline shell, has ultra-low overall skin electrode contact impedance, ultra-thinness, flexibility, and good adhesion and compliance to fine lines of human skin, and can be stored for a long time. The dual-conductive fiber network structure material provided by the present invention solves the problems of large signal-to-noise ratio and poor compliance and stability of electrodes of skin-contact devices in the prior art, and the preparation method provided by the present invention solves the problem of difficulty in spinning the chitosan solution and the polyacrylonitrile-polyaniline solution in the prior art and reconciles the problems of stability of chitosan gel fibers and conductivity requirements of protonic acid-doped polyaniline.

### DESCRIPTION OF THE DRAWINGS

FIG. 1 is a scanning electron microscope (SEM) image of a polyacrylonitrile-chitosan fiber pad obtained in step (2) in embodiment 1 of the present invention;

FIG. 2 is an SEM image of a sulfuric acid-doped polyacrylonitrile@polyaniline-chitosan@polyaniline dual network core-shell structure electrode obtained in step (3) in embodiment 1 of the present invention;

FIG. 3 is a photo of compliance characterization of a sulfuric acid-doped polyacrylonitrile@polyaniline-chitosan@polyaniline dual network core-shell structure electrode obtained in step (3) in embodiment 1 of the present invention attached to the back of a wrist;

FIG. 4 shows measurement and characterization results of skin electrode contact impedance of a PAN@PANI‑Chitosan@PANI dual-core-shell structure spinning electrode prepared in embodiment 1, a PAN@PANI single-core-shell structure spinning electrode prepared in reference example 1 and a commercial electrode prepared in reference example 2 of the present invention.

### DETAILED DESCRIPTION OF THE INVENTION

The present invention discloses a dual-conductive fiber network structure material and a preparation method and application thereof. Those skilled in the art can learn from the content of the present invention to improve the realization of technological parameters appropriately. It should be particularly noted that all similar substitutions and modifications are apparent to those skilled in the art and are considered to be included in the present invention. The method and application of the present invention is described through preferred embodiments. Related personnel can obviously modify or appropriately change and combine the method and application described herein without departing from the content, spirit and scope of the present invention to realize and apply the technology of the present invention.

The present invention will be further described below in combination with the embodiments.

### Embodiment 1

(1) A specific preparation method for polyaniline particles is as follows:

① Dissolving 4.0 g of aniline in 50 mL of 1 M hydrochloric acid solution, and dissolving 12.3 g of ammonium persulfate in 50 mL of 1 M hydrochloric acid solution. Mixing the two solutions at room temperature. After 2 h, carrying out suction filtration and drying to obtain doped polyaniline;

② Adding 150 mL of saturated aqueous ammonia, standing for 24 h, and deprotonating polyaniline particles;

③ Carrying out suction filtration of deprotonated polyaniline to remove excess aqueous ammonia, and drying.

(2) Specific steps of preparing a polyacrylonitrile-chitosan fiber electrospinning pad are as follows:

① Installing an inclined air outlet of a fixed drying-type dehumidifier, with a preferred angle of 30°;

② Designing and preparing dual-phase conjugate spinneret spinning, with the spacing between spinneret nozzles of approximately 1 cm, to prevent the two spinning solutions from prematurely condensing due to mutual effect at the exit;

③ Preparing a DMF solution with a polyacrylonitrile (Mv=150000) mass fraction of 10 wt%;

④ Dropping 0.1 g of NMP in 0.5 g of polyaniline synthesized in step (1), and mixing with water to prepare an NMP solution with a polyaniline mass fraction of 10 wt%;

⑤ Mixing ④ and ③ at 25% (a proportion of ④ in total weight) to obtain a polyacrylonitrile/polyaniline dispersion liquid to be spun;

⑥ Preparing a 10 wt% aqueous solution of PVA (with a viscosity coefficient of 54-66 mPa.s-1);

⑦ Preparing 90 wt% acetic acid as a solution to prepare an acetic acid solution of 3 wt% chitosan (with a deacetylation degree greater than 99% and low polymerization degree);

⑧ Mixing ⑥ and ⑦ at a weight ratio of 0.5 g:12.5 g to obtain a chitosan/PVA solution to be spun;

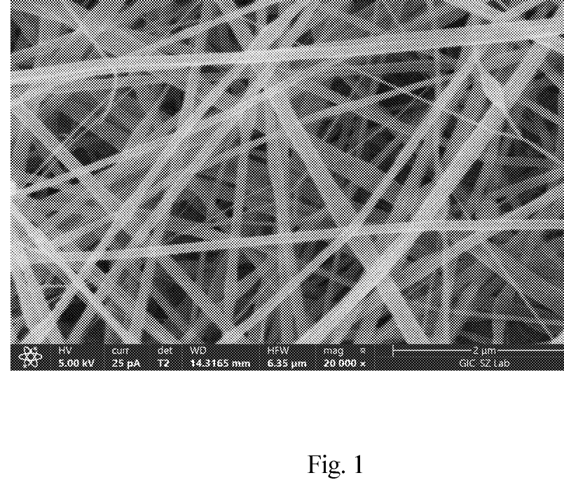

⑨ Carrying out two-component spinning of ⑤ and ⑧ at a volume ratio of 1:1, with a collecting barrel about 15 cm away from the spinnerets, the injection rate of 8 µL/min for a single component and a voltage of 25 kV, thus obtaining a polyacrylonitrile-chitosan fiber pad, as shown in FIG. 1. FIG. 1 is an SEM image of the polyacrylonitrile-chitosan fiber pad obtained in step (2) in embodiment 1 of the present invention.

(3) Specific steps of preparing of a sulfuric acid-doped polyacrylonitrile@polyaniline-chitosan@polyaniline dual network core-shell structure spinning electrode are as follows;

① Immersing the polyacrylonitrile-chitosan fiber pad obtained in step (2) in a 2 M sodium hydroxide solution for 2 min to cross-link and reinforce chitosan fibers;

② Preparing 100 mL of 3.06 wt% ammonium persulfate solution, 100 mL of 11.05 wt% sulfuric acid solution and 50 mL of 2 wt% aniline solution respectively, and the solvents of the solutions are all 50% ethanol aqueous solutions. Cooling the solutions in a 0‑2℃ refrigerator, and then mixing the solutions;

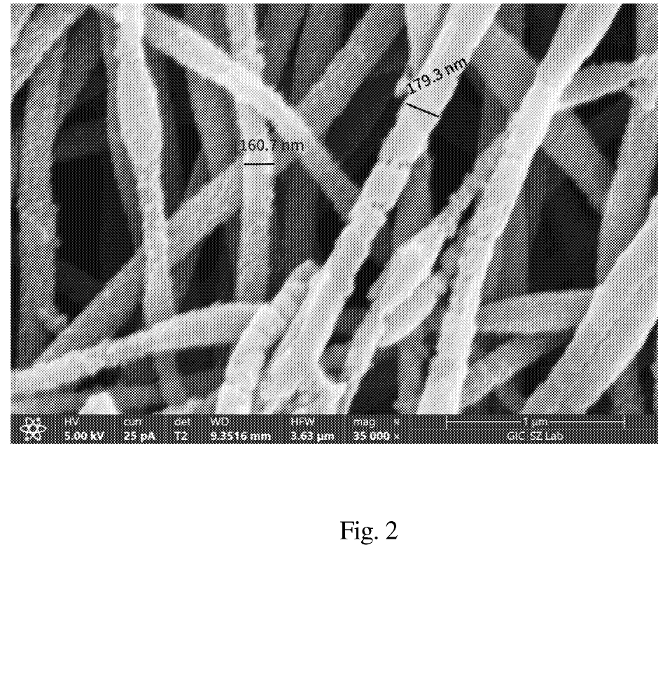

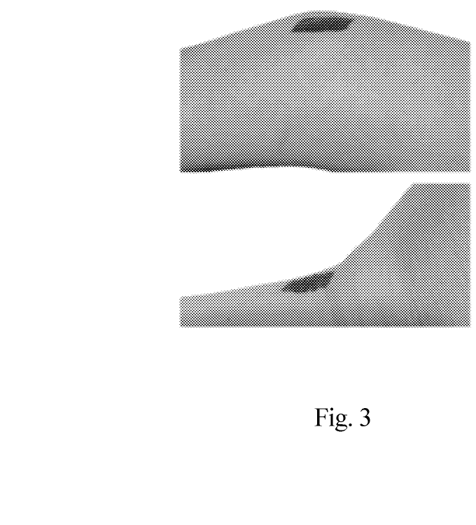

③ Placing the cross-linked and reinforced polyacrylonitrile-chitosan fiber pad in the mixed solution obtained in ②, and continuing to stand in the 0‑2℃ refrigerator for24 h, thus obtaining the sulfuric acid-doped polyacrylonitrile@polyaniline-chitosan@polyaniline dual network core-shell structure spinning electrode (PAN@PANI‑Chitosan@PANI dual-core-shell structure spinning electrode). As shown in FIG. 2, FIG. 2 is an SEM image of the sulfuric acid-doped polyacrylonitrile@polyaniline-chitosan@polyaniline dual network core-shell structure electrode obtained in step (3) in embodiment 1 of the present invention. As shown in FIG. 3, FIG. 3 is a photo of compliance characterization of the sulfuric acid-doped polyacrylonitrile@polyaniline-chitosan@polyaniline dual network core-shell structure electrode obtained in step (3) in embodiment 1 of the present invention attached to the back of a wrist.

### Reference example 1

A polyacrylonitrile/polyaniline dispersion liquid to be spun which is the same as that in embodiment 1 is prepared and is subjected to single-component spinning according to the same spinning conditions as in embodiment 1 to obtain a polyacrylonitrile fiber electrospinning pad; and the obtained polyacrylonitrile fiber electrospinning pad is cross-linked and reinforced and is doped with sulfuric acid according to the same method as in embodiment 1 to obtain a PAN@PANI single-core-shell structure spinning electrode.

### Reference example 2

A commercial electrode is an Ag/AgCl gel electrode.

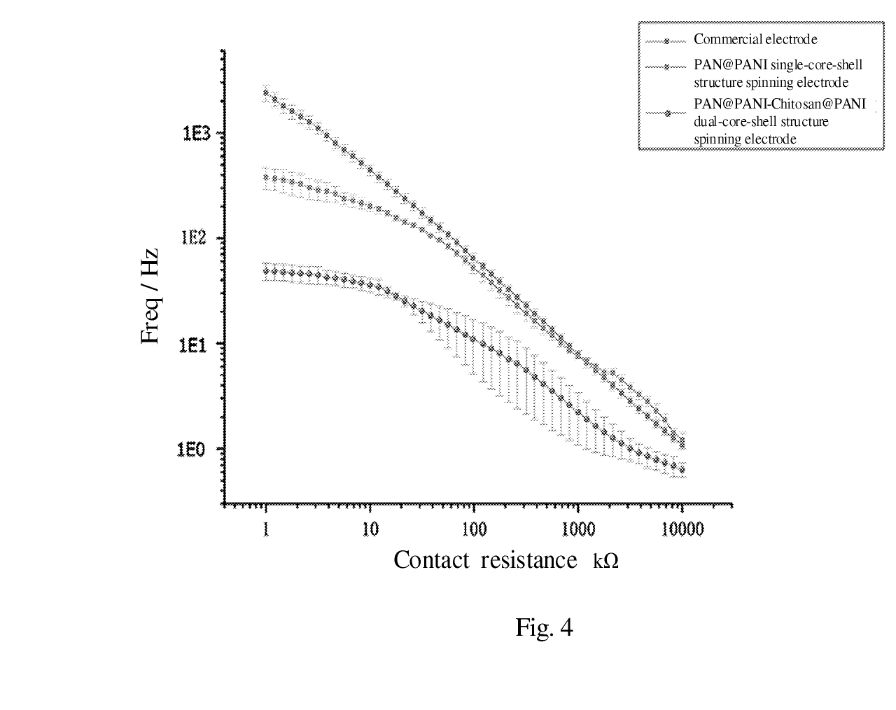

Skin electrode contact impedance is measured and characterized for the electrodes of embodiment 1, reference example 1 and reference example 2, and the results are shown in FIG. 4. FIG. 4 shows measurement and characterization results of skin electrode contact impedance of the PAN@PANI‑Chitosan@PANI dual-core-shell structure spinning electrode prepared in embodiment 1, the PAN@PANI single-core-shell structure spinning electrode prepared in reference example 1 and the commercial electrode prepared in reference example 2 of the present invention.

The above are just preferred embodiments of the present invention, but the protection scope of the present invention is not limited thereto. Any equivalent replacement or change made by those skilled in the art familiar with the technical field within the technical scope disclosed by the present invention shall be covered within the protection scope of the present invention.

1. A dual-conductive fiber network structure material, comprising:
a conductive fiber network, comprised of polyacrylonitrile, deprotonated polyaniline, chitosan and polyvinyl alcohol (PVA);
sulfuric acid-doped polyaniline cladded on the conductive fiber network.

2. A preparation method for a dual-conductive fiber network structure material, comprising the following steps:
S1) electrospinning a first spinning solution and a second spinning solution at an exit spacing of 0.9-3.0 cm;
the first spinning solution comprises polyacrylonitrile and deprotonated polyaniline;
the second spinning solution comprises chitosan and PVA;
S2) after curing a material obtained in step S1) in an alkaline solution, cladding sulfuric acid-doped polyaniline on a surface of the material to obtain a dual-conductive fiber network structure material.

3. The preparation method as claimed in claim 2, wherein in step S1), the first spinning solution is obtained from a polyacrylonitrile solution with a polyacrylonitrile mass fraction of 5 wt%-20 wt% and a deprotonated polyaniline dispersion liquid with a deprotonated polyaniline mass fraction of 8 wt%-15 wt%;
a weight ratio of the deprotonated polyaniline dispersion liquid in the first spinning solution is 10%-35%.

4. The preparation method as claimed in claim 2, wherein in step S1), the second spinning solution is obtained from a PVA solution and a chitosan solution with a chitosan mass fraction of 3 wt%-4 wt%;
a weight ratio of the PVA solution to the chitosan solution is 0.5:(8-15).

5. The preparation method as claimed in claim 2, wherein in step S1), a spinning speed of the electrospinning is 7-30 µL/min.

6. The preparation method as claimed in claim 2, wherein in step S1), a spinning voltage of the electrospinning is 19-30 kV.

7. The preparation method as claimed in claim 2, wherein in step S1), when the electrospinning is carried out, a spinning solution jet is dried with hot air at an angle of 20-50°.

8. The preparation method as claimed in claim 2, wherein in step S1), the deprotonated polyaniline is prepared by the following method:
obtaining doped polyaniline from ammonium persulfate, hydrochloric acid and aniline, and deprotonating the doped polyaniline in aqueous ammonia to obtain deprotonated polyaniline.

9. The preparation method as claimed in claim 2, wherein step S2) is specifically as follows:
after curing the material obtained in step S1) in a sodium hydroxide solution, letting the cured material stand in a mixed solution of ammonium persulfate, sulfuric acid and aniline at 0-2℃ to obtain a dual-conductive fiber network structure material.

10. Application of the dual-conductive fiber network structure material of claim 1 in the preparation of electrodes.

11. Application of the dual-conductive fiber network structure material obtained by the preparation method of claim 2 in the preparation of electrodes.

12. Application of the dual-conductive fiber network structure material obtained by the preparation method of claim 3 in the preparation of electrodes.

13. Application of the dual-conductive fiber network structure material obtained by the preparation method of claim 4 in the preparation of electrodes.

14. Application of the dual-conductive fiber network structure material obtained by the preparation method of claim 5 in the preparation of electrodes.

15. Application of the dual-conductive fiber network structure material obtained by the preparation method of claim 6 in the preparation of electrodes.

16. Application of the dual-conductive fiber network structure material obtained by the preparation method of claim 7 in the preparation of electrodes.

17. Application of the dual-conductive fiber network structure material obtained by the preparation method of claim 8 in the preparation of electrodes.

18. Application of the dual-conductive fiber network structure material obtained by the preparation method of claim 9 in the preparation of electrodes.
